(19) United States
(12) Patent Application Publication
(10) Pub. No.: US 2010/0056544 A1
(43) Pub. Date: Mar. 4, 2010

(54) Salts with CRTH2 Antagonist Activity

(75) Inventor: James Matthew Lovell, Oxon (GB)

Correspondence Address: STERNE, KESSLER, GOLDSTEIN & FOX P.L.L.C., 1100 NEW YORK AVENUE, N.W., WASHINGTON, DC, 20005, US

(73) Assignee: Oxagen Limited, Abingdon (GB)

(21) Appl. No.: 12/293,504

(22) PCT Filed: Mar. 22, 2007
(86) PCT No.: PCT/GB07/01038

§ 371 Date: Oct. 22, 2009

(51) Int. Cl. A61K 31/497; C07D 403/02; C07D 209/04; A61K 31/40; A61P 11/06

(52) U.S. Cl. 514/254.09; 544/373; 548/510; 514/412

(57) ABSTRACT

The potassium, sodium, ammonium, lysine, diethylamine, tromethamine (TRIS), piperazine, ethylenediamine and ethanolamine salt of a compound of general formula (I): wherein R¹ is halo or cyano; R² is C₁-C₄ alkyl; and R³ is quinolyl or phenyl substituted with methane sulfonyl; can be synthesised by a novel method and are substantially more soluble than the parent free acids in a range of solvents.

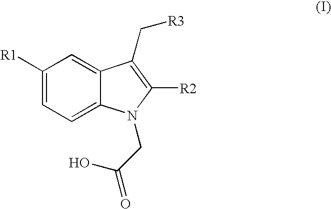

The present invention relates to compounds which are useful as pharmaceuticals. In particular, the invention relates to salts which are particularly soluble in a range of solvents. The invention also relates to methods for preparing these salts, compositions containing them and their use in the treatment and prevention of allergic diseases such as asthma, allergic rhinitis and atopic dermatitis and other inflammatory diseases mediated by prostaglandin D₂ (PGD₂) acting at the CRTH2 receptor on cells including eosinophils, basophils and Th2 lymphocytes.

PGD₂ is an eicosanoid, a class of chemical mediator synthesised by cells in response to local tissue damage, normal stimuli or hormonal stimuli or via cellular activation pathways. Eicosanoids bind to specific cell surface receptors on a wide variety of tissues throughout the body and mediate various effects in these tissues. PGD₂ is known to be produced by mast cells, macrophages and Th2 lymphocytes and has been detected in high concentrations in the airways of asthmatic patients challenged with antigen (Murray et al, (1986), N. Engl. J. Med. 315: 800-804). Instillation of PGD₂ into airways can provoke many features of the asthmatic response including bronchoconstriction (Hardy et al, (1984)N. Engl. J. Med. 311: 209-213; Sampson et al, (1997) Thorax 52: 513-518) and eosinophil accumulation (Emery et al, (1989) J. Appl. Physiol. 67: 959-962).

The potential of exogenously applied PGD₂ to induce inflammatory responses has been confirmed by the use of transgenic mice overexpressing human PGD₂ synthase which exhibit exaggerated eosinophilic lung inflammation and Th2 cytokine production in response to antigen (Fujitani et al, (2002) J. Immunol. 168: 443-449).

The first receptor specific for PGD₂ to be discovered was the DP receptor which is linked to elevation of the intracellular levels of cAMP. However, PGD₂ is thought to mediate much of its proinflammatory activity through interaction with a G protein-coupled receptor termed CRTH2 (chemoattractant receptor-homologous molecule expressed on Th2 cells) which is expressed by Th2 lymphocytes, eosinophils and basophils (Hirai et al, (2001) J. Exp. Med. 193: 255-261, and EP0851030 and EP-A-1211513 and Bauer et al, EP-A-1170594). It seems clear that the effect of PGD₂ on the activation of Th2 lymphocytes and eosinophils is mediated through CRTH2 since the selective CRTH2 agonists 13,14 dihydro-15-keto-PGD₂ (DK-PGD₂) and 15R-methyl-PGD₂ can elicit this response and the effects of PGD₂ are blocked by an anti-CRTH2 antibody (Hirai et al, 2001; Monneret et al, (2003) J. Pharmacol. Exp. Ther. 304: 349-355). In contrast, the selective DP agonist BW245C does not promote migration of Th2 lymphocytes or eosinophils (Hirai et al, 2001; Gervais et al, (2001) J. Allergy Clin. Immunol. 108: 982-988). Based on this evidence, antagonising PGD₂ at the CRTH2 receptor is an attractive approach to treat the inflammatory component of Th2-dependent allergic diseases such as asthma, allergic rhinitis and atopic dermatitis.

EP-A-1170594 suggests that the method to which it relates can be used to identify compounds which are of use in the treatment of allergic asthma, atopic dermatitis, allergic rhinitis, autoimmune disease, reperfusion injury and a number of inflammatory conditions, all of which are mediated by the action of PGD₂ at the CRTH2 receptor.

Compounds which bind to CRTH2 are taught in WO-A-03066046 and WO-A-03066047. These compounds are not new but were first disclosed, along with similar compounds, in GB 1356834, GB 1407658 and GB 1460348, where they were said to have anti-inflammatory, analgesic and antipyretic activity. WO-A-03066046 and WO-A-03066047 teach that the compounds to which they relate are modulators of CRTH2 receptor activity and are therefore of use in the treatment or prevention of obstructive airway diseases such as asthma, chronic obstructive pulmonary disease (COPD) and a number of other diseases including various conditions of bones and joints, skin and eyes, GI tract, central and peripheral nervous system and other tissues as well as allograft rejection.

PL 65781 and JP 43-24418 also relate to indole derivatives which are similar in structure to indomethacin and, like indomethacin, are said to have anti-inflammatory and antipyretic activity. Thus, although this may not have been appreciated at the time when these documents were published, the compounds they describe are COX inhibitors, an activity which is quite different from that of the compounds of the present invention. Indeed, COX inhibitors are contraindicated in the treatment of many of the diseases and conditions, for example asthma and inflammatory bowel disease for which the compounds of the present invention are useful, although they may sometimes be used to treat arthritic conditions.

The present inventors have discovered a series of indole acetic acids which are particularly active antagonists of PGD2 at the CRTH2 receptor.

WO-A-9950268, WO-A-0032180, WO-A-0151849 and WO-A-0164205 all relate to indole acetic acids. However, these compounds are said to be aldose reductase inhibitors useful in the treatment of diabetes mellitus (WO-A-9950268, WO-A-0032180 and WO-A-0164205) or hypouricemic agents (WO-A-0151849).

U.S. Pat. No. 4,363,912 also relates to indole acetic acids which are said to be inhibitors of thromboxane synthetase and to be useful in the treatment of conditions such as thrombosis, ischaemic heart disease and stroke. The compounds are all substituted with a pyridyl group.

WO-A-9603376 relates to compounds which are said to be sPLA₂ inhibitors which are useful in the treatment of bronchial asthma and allergic rhinitis. These compounds are amides or hydrazides rather than carboxylic acids.

JP 2001247570 relates to a method of producing a 3-benzothiazolylmethyl indole acetic acid, which is said to be an aldose reductase inhibitor.

U.S. Pat. No. 4,859,692 relates to compounds which are said to be leukotriene antagonists useful in the treatment of conditions such as asthma, hay fever and allergic rhinitis as well as certain inflammatory conditions such as bronchitis, atopic and ectopic eczema. However, J. Med. Chem., 6(33), 1781-1790 (1990), which has the same authors as this prior patent application, teaches that compounds with an acetic acid group on the indole nitrogen do not have significant peptidoleukotriene activity. In view of this, it is most surprising that the compounds of the present invention, which all have an acetic acid group on the indole nitrogen, are useful for treating conditions such as asthma, hay fever and allergic rhinitis.

U.S. Pat. No. 4,273,782 is directed imidazole substituted indole acetic acids which are said to be useful in the treatment of conditions such as thrombosis, ischaemic heart disease, stroke, transient ischaemic attack, migraine and the vascular complications of diabetes. There is no mention in the document of conditions mediated by the action of PGD₂ at the CRTH2 receptor.

U.S. Pat. No. 3,557,142 relates to 3-substituted-1-indole carboxylic acids and esters which are said to be useful in the treatment of inflammatory conditions.

WO-A-03/097598 relates to compounds which are CRTH2 receptor antagonists. They do not have an aromatic substituent.

Cross et al, J. Med. Chem. 29, 342-346 (1986) relates to a process for preparing imidazole-substituted indole acetic acids from the corresponding esters. The compounds to which it relates are said to be inhibitors of thromboxane synthetase.

EP-A-0539117 relates to indole acetic acid derivatives which are said to be leukotriene antagonists.

US 2003/0153751 relates to compounds which are sPLA₂ inhibitors. All of the exemplified compounds have bulky substituents at the 2- and 5-positions of the indole system.

US 2004/011648 discloses indole acetic acid derivatives which are inhibitors of PAI-1. There is no suggestion that the compounds might have CRTH2 antagonist activity.

WO 2004/058164 relates to compounds which are said to be asthma and allergic inflammation modulators. There is no demonstration of any activity for indole acetic acid derivatives.

Compounds which bind to the CRTH2 receptor are disclosed in WO-A-03/097042 and WO-A-03/097598. These compounds are indole acetic acids and in WO-A-03/097042 the indole system is fused at the 2-3 positions to a 5-7 membered carbocyclic ring. In WO-A-03/097598 there is a pyrrolidine group at the indole 3-position.

WO-A-03/101981 and WO-A-03/101961 both relate to compound which are said to be CRTH2 antagonists and which are indole acetic acids with an —S— or —SO₂— group linked to the indole 3-position.

In our patent application WO-A-2005/044260, we disclose indole carboxylic acids which are particularly active CRTH2 antagonists. The document also teaches salts of these compounds and specifically the lithium salts which were intermediates in the preparation of the free acids.

However, we have now discovered that certain salts of some of the compounds of WO-A-2005/044260 have surprising properties. In order for a compound to be useful in medicine, it is advantageous to be able to dissolve that compound in an aqueous solvent. However, when we attempted to dissolve the free acids of WO-A-2005/044260 in a wide range of solvents, we found that they were at best sparingly soluble in any of the solvents we used, including water. It would be expected that a salt would be more soluble in an aqueous solvents than the parent free acid but the present inventors have discovered that certain salts of some compounds disclosed in WO-A-2005/044260 have unexpectedly high solubility in aqueous media. This high solubility does not extend to all of the salts of the selected compounds and this is also unexpected.

Therefore, in a first aspect of the present invention there is provided a potassium, sodium, ammonium, lysine, diethylamine, tromethamine (TRIS), piperazine, ethylenediamine or ethanolamine salt of a compound of general formula (I):

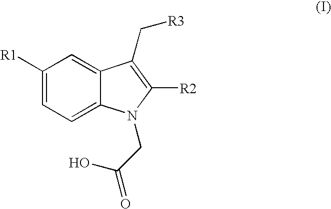

wherein R¹ is halo or cyano;
R² is C₁-C₄ alkyl; and
R³ is quinolyl or phenyl substituted with methane sulfonyl.

It is expected that salts would be more soluble than the free acid compounds from which they are derived but the solubility of the salts of the present invention in water ranged from 65 to about 1700 times greater than that of the parent compound and this degree of improvement in solubility is unexpected. The solubility of the salts in other solvents was also much greater than that of the parent free acids.

Particularly soluble salts of the present invention are the potassium salt the sodium salt, the ethanolamine salt and the piperazine salt.

In preferred compounds of general formula (I), independently or in any combination:

R¹ is fluoro;
R² is methyl;
R³ is 2-quinolyl or 4-methanesulfonylphenyl.

Particularly preferred compounds of the present invention are the potassium, sodium, ammonium, lysine, diethylamine, TRIS, piperazine, ethylenediamine or ethanolamine salts of:
(5-fluoro-2-methyl-3-quinolin-2-ylmethyl-indol-1-yl)acetic acid (Compound 1); and
[5-fluoro-3-(4-methanesulfonylbenzyl)-2-methyl-indol-1-yl]acetic acid (Compound 2).

As discussed above, salts of the compounds of general formula (I) are taught in WO-A-2005/044260 and may be prepared by the methods set out in that document. In WO-A-2005/044260, the compounds of general formula (I) were prepared initially as lithium salts by the hydrolysis of an ester using lithium hydroxide. It is also possible to prepare other salts of all of the compounds taught in WO-A-2005/044260 by the hydrolysis of the corresponding ester with a selected base, for example ammonium hydroxide, potassium hydroxide and sodium hydroxide.

However, once the free acid of general formula (I) has been obtained, it has proved difficult to convert it back to a salt. Usually, salts can be prepared by dissolving a free acid in an appropriate solvent and adding a base and it is, indeed, possible to prepare small amounts of salts of the compounds of general formula (I) in this way. However, because the free acids of general formula (I) are only sparingly soluble in most solvents, it has not proved to be viable to use this method of salt preparation on a large scale. It has therefore been necessary for the inventors to develop a modified method for the large scale preparation of the salts of the present invention.

Therefore, in a second aspect of the invention, there is provided a process for the preparation of a potassium, sodium, ammonium, lysine, diethylamine, tromethamine (TRIS), piperazine, ethylenediamine or ethanolamine salt of a compound of general formula (I) as defined above, the process comprising the steps of:
a) adding to the parent free acid of general formula (I) about 8 to 20 volumes of acetonitrile and about 2 to 3 molar equivalents of an appropriate base;
b) if necessary adding to the mixture sufficient water to dissolve the base;
c) heating the mixture to between 40 and 60° C.;
d) allowing the mixture to cool to about 15 to 25° C.; and
e) collecting the precipitated salt.

Appropriate bases for use in preparing the salts of the invention are: ammonium hydroxide, lysine, potassium hydroxide, sodium hydroxide, diethylamine, ethanolamine, ethylenediamine, piperazine and tromethamine (TRIS).

It is preferred that, in step (a), about 10 volumes of acetonitrile are added to the parent free acid and that about 2 molar equivalents of base are used.

The precipitated salt may be collected by filtration and may be washed using an appropriate solvent such as acetonitrile.

Compounds of general formula (I) may be prepared as set out in our co-pending application WO-A-2005/044260 and a specific method for particular compounds of general formula (I) is set out in the examples below.

As mentioned above, the salts of the present invention are surprisingly soluble in a range of aqueous solvents and therefore, in a further aspect of the present invention, there is provided an aqueous solution comprising at least 3 mg/ml of a salt selected from the potassium, sodium, ammonium, lysine, diethylamine, tromethamine (TRIS), piperazine, ethylenediamine or ethanolamine salt of a compound of general formula (I). The aqueous solution preferably comprises at least 10 mg/ml of a salt selected from the potassium, sodium, piperazine or ethanolamine salt of a compound of general formula (I) and more preferably it comprises at least 30 mg/ml of the potassium, sodium, piperazine or ethanolamine salt of a compound of general formula (I).

The salts of the compounds general formula (I) are useful in a method for the treatment of diseases or conditions mediated by the action of PGD₂ at the CRTH2 receptor, the method comprising administering to a patient in need of such treatment an appropriate amount of a salt of a compound general formula (I).

Therefore, in a further aspect of the invention, there is provided a potassium, sodium, ammonium, lysine, diethylamine, tromethamine (TRIS), piperazine, ethylenediamine or ethanolamine salt of a compound of general formula (I) for use in medicine.

The salts are particularly useful for the treatment of particularly for use in the treatment or prevention of diseases and conditions mediated by PGD₂ at the CRTH2 receptor.

Such diseases and conditions include allergic asthma, perennial allergic rhinitis, seasonal allergic rhinitis, atopic dermatitis, contact hypersensitivity (including contact dermatitis), conjunctivitis, especially allergic conjunctivitis, eosinophilic bronchitis, food allergies, eosinophilic gastroenteritis, inflammatory bowel disease, ulcerative colitis and Crohn's disease, mastocytosis and also other PGD₂-mediated diseases, for example autoimmune diseases such as hyper IgE syndrome and systemic lupus erythematus, psoriasis, acne, multiple sclerosis, allograft rejection, reperfusion injury, chronic obstructive pulmonary disease, as well as rheumatoid arthritis, psoriatic arthritis and osteoarthritis; and also neurodegenerative diseases such as Alzheimer's disease, Parkinson's disease, stroke and amyotrophic lateral sclerosis.

In a further aspect of the invention, there is provided the use of a potassium, sodium, ammonium, lysine, diethylamine, tromethamine (TRIS), piperazine, ethylenediamine or ethanolamine salt of a compound general formula (I) in the preparation of an agent for the treatment of allergic asthma, perennial allergic rhinitis, seasonal allergic rhinitis, atopic dermatitis, contact hypersensitivity (including contact dermatitis), conjunctivitis, especially allergic conjunctivitis, eosinophilic bronchitis, food allergies, eosinophilic gastroenteritis, inflammatory bowel disease, ulcerative colitis and Crohn's disease, mastocytosis and also other PGD₂-mediated diseases, for example autoimmune diseases such as hyper IgE syndrome and systemic lupus erythematus, psoriasis, acne, multiple sclerosis, allograft rejection, reperfusion injury, chronic obstructive pulmonary disease, as well as rheumatoid arthritis, psoriatic arthritis and osteoarthritis and neurodegenerative diseases such as Alzheimer's disease, Parkinson's disease, stroke and amyotrophic lateral sclerosis.

The salts of compounds of general formula (I) must be formulated in an appropriate manner depending upon the diseases or conditions they are required to treat.

Therefore, in a further aspect of the invention there is provided a pharmaceutical composition comprising a potassium, sodium, ammonium, lysine, diethylamine, tromethamine (TRIS), piperazine, ethylenediamine or ethanolamine salt of a compound of general formula (I) together with a pharmaceutical excipient or carrier. Other active materials may also be present, as may be considered appropriate or advisable for the disease or condition being treated or prevented.

The carrier, or, if more than one be present, each of the carriers, must be acceptable in the sense of being compatible with the other ingredients of the formulation and not deleterious to the recipient.

The formulations include those suitable for oral, rectal, nasal, bronchial (inhaled), topical (including eye drops, buccal and sublingual), vaginal or parenteral (including subcutaneous, intramuscular, intravenous and intradermal) administration and may be prepared by any methods well known in the art of pharmacy.

The composition may be prepared by bringing into association the above defined active agent with the carrier. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a salt of a compound of general formula (I) in conjunction or association with a pharmaceutically or veterinarily acceptable carrier or vehicle.

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

For topical application to the skin, a salt of a compound of general formula (I) may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

Salts of compound of general formula (I) may be used for the treatment of the respiratory tract by nasal, bronchial or buccal administration of, for example, aerosols or sprays which can disperse the pharmacological active ingredient in the form of a powder or in the form of drops of a solution or suspension. Pharmaceutical compositions with powder-dispersing properties usually contain, in addition to the active ingredient, a liquid propellant with a boiling point below room temperature and, if desired, adjuncts, such as liquid or solid non-ionic or anionic surfactants and/or diluents. Pharmaceutical compositions in which the pharmacological active ingredient is in solution contain, in addition to this, a suitable propellant, and furthermore, if necessary, an additional solvent and/or a stabiliser. Instead of the propellant, compressed air can also be used, it being possible for this to be produced as required by means of a suitable compression and expansion device.

Parenteral formulations will generally be sterile.

Typically, the dose of the salt will be about 0.01 to 100 mg/kg; so as to maintain the concentration of drug in the plasma at a concentration effective to inhibit PGD₂ at the CRTH2 receptor. The precise amount of a salt of a compound of general formula (I) which is therapeutically effective, and the route by which such salt is best administered, is readily determined by one of ordinary skill in the art by comparing the blood level of the agent to the concentration required to have a therapeutic effect.

The potassium, sodium, ammonium, lysine, diethylamine, tromethamine (TRIS), piperazine, ethylenediamine or ethanolamine salts of compounds of general formula (I) may be used in combination with one or more active agents which are useful in the treatment of the diseases and conditions listed above, although these active agents are not necessarily inhibitors of PGD₂ at the CRTH2 receptor.

Therefore, the pharmaceutical composition described above may additionally contain one or more of these active agents.

There is also provided the use of a potassium, sodium, ammonium, lysine, diethylamine, tromethamine (TRIS), piperazine, ethylenediamine or ethanolamine salt of a compound of general formula (I) in the preparation of an agent for the treatment of diseases and conditions mediated by PGD₂ at the CRTH2 receptor, wherein the agent also comprises an additional active agent useful for the treatment of the same diseases and conditions.

These additional active agents which may have a completely different mode of action include existing therapies for allergic and other inflammatory diseases including:

β2 agonists such as salmeterol;
corticosteroids such as fluticasone;
antihistamines such as loratidine;
leukotriene antagonists such as montelukast;
anti-IgE antibody therapies such as omalizumab;
anti-infectives such as fusidic acid (particularly for the treatment of atopic dermatitis);
anti-fungals such as clotrimazole (particularly for the treatment of atopic dermatitis);
immunosuppressants such as tacrolimus and particularly pimecrolimus in the case of inflammatory skin disease.

CRTH2 antagonists may also be combined with therapies that are in development for inflammatory indications including:

other antagonists of PGD₂ acting at other receptors, such as DP antagonists;
inhibitors of phosphodiesterase type 4 such as cilonilast;
drugs that modulate cytokine production such as inhibitors of TNFα converting enzyme (TACE);
drugs that modulate the activity of Th2 cytokines IL-4 and IL-5 such as blocking monoclonal antibodies and soluble receptors;
PPAR-γ agonists such as rosiglitazone;
5-lipoxygenase inhibitors such as zileuton.

In yet a further aspect of the invention, there is provided a product comprising a potassium, sodium, ammonium, lysine, diethylamine, tromethamine (TRIS), piperazine, ethylenediamine or ethanolamine salt of general formula (I) and one or more of the agents listed above as a combined preparation for simultaneous, separate or sequential use in the treatment of a disease or condition mediated by the action of PGD₂ at the CRTH2 receptor.

The invention will now be described in greater detail with reference to the following non limiting examples and the drawing.

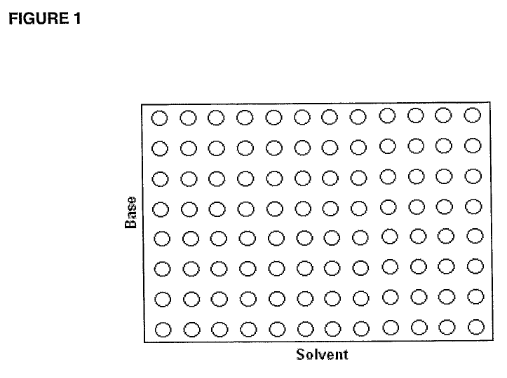

FIG. 1 is a representation of a 96 well plate in which each line in the x direction contains a different base except for the 8^{th} line which was left blank and in which different potential crystallizing solvents can be added to each row in the y direction.

In the Examples, the following abbreviations are used.

| | |
|---|---|
| IPA—2-propanol | DMF—N,N-dimethylformamide |
| DMSO—dimethylsulfoxide | EtOAc—ethyl acetate |
| NMP—N-methylpyrrolidine | MeOH—methanol |
| TBME—tert-butylmethylether | DCM—dichloromethane |

### EXAMPLE 1

### Synthesis of (5-fluoro-2-methyl-3-quinolin-2-ylmethyl-indol-1-yl)-acetic acid (Compound 1)

### Stage 1: Synthesis of ethyl-(5-fluoro-2-methylindolyl-1-acetate)

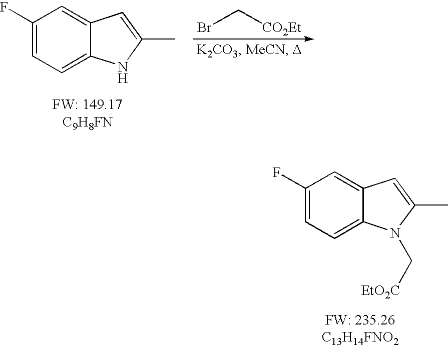

5-Fluoro-2-methylindole (0.45 Kg, 3.017 mol, 1.0 wt), powdered potassium carbonate (1.251 Kg, 9.05 mol, 2.78 wt) and acetonitrile (9.0 L, 20 vol) were charged to a 20 L flange flask at 15 to 25° C. Ethyl bromoacetate (0.671 L, 2.67 mol, 1.49 vol) was added and the resulting suspension heated to and maintained at reflux for 18 h after which time in-process check analysis by ¹H NMR¹ indicated 87% conversion. A further charge of ethyl bromoacetate (0.333 L, 1.32 mol, 0.74 vol) and powdered potassium carbonate (0.626 Kg, 4.53 mol, 1.39 wt) was made and reflux conditions established for a further 6 hours. In-process check by ¹H NMR¹ analysis indicated 98.4% conversion. The flask contents were allowed to cool to 15 to 25° C. over 16 hours. The solids were removed by filtration and the filter-cake washed with acetonitrile (2×1 L, 2×2 vol). The combined filtrates were concentrated to dryness under vacuum at up to 40° C. (water bath) to provide crude Stage 1 as a brown oil (1.286 Kg). The crude product was purified by dry flash chromatography using a gradient elution from heptanes to heptanes:toluene to toluene to give ethyl-(5-fluoro-2-methylindolyl-1-acetate) as an off-white solid (0.573 Kg, 80.7% theoretical, corrected for residual toluene). Mixed fractions were re-chromatographed as appropriate. ¹Reaction sampled, the sample concentrated, the residue taken up in D₆-DMSO, filtered and the ¹H NMR spectrum recorded

### Stage 2: Synthesis of (5-Fluoro-2-methyl-3-quinolin-2-ylmethylindo-1-yl)-acetic acid ethyl ester

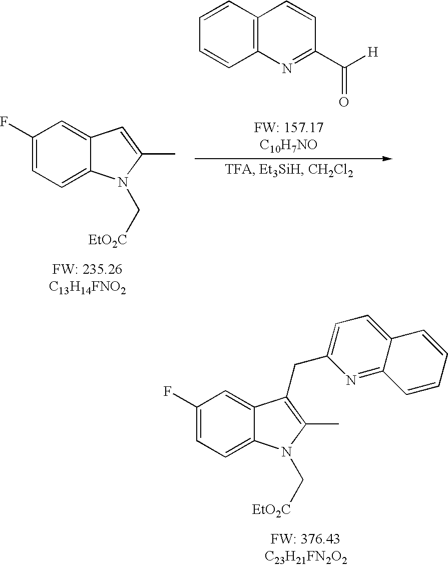

Ethyl-(5-fluoro-2-methylindolyl-1-acetate) (0.573 Kg, 2.44 mol, 1.0 wt) and quinoline-2-carboxaldehyde (0.418 Kg, 2.66 mol, 0.735 wt) as a solution in dichloromethane (5.73 L, 10 vol) at 0 to 5° C. were treated with triethylsilane (1.369 L, 8.51 mol, 2.39 vol) followed by the drop-wise addition of trifluoroacetic acid (0.561 L, 7.28 mol, 0.98 vol) at 0 to 10° C. The resulting dark red solution was warmed to and maintained at reflux for 3 h after which time in-process check analysis by ¹H NMR² indicated reaction completion. The reaction was cooled to 15 to 25° C. and quenched by the addition of saturated sodium hydrogen carbonate solution (11.5 L, 20 vol) over 0.5 h (note: foaming and gas evolution). The layers were separated, the aqueous layer extracted with dichloromethane (1×2.8 L, 1×5.0 vol), the combined organics washed with 20% w/w aqueous sodium chloride solution (1×3.0 L, 1×5 vol) and dried over sodium sulfate (0.6 Kg, 1.05 wt). The suspension was filtered, the filter-cake washed with dichloromethane (2×0.6 L, 2×1.05 vol) and the combined filtrates concentrated under vacuum at up to 40° C. (water bath) to afford (5-fluoro-2-methyl-3-quinolin-2-ylmethylindo-1-yl)-acetic acid ethyl ester as a brown oily solid (1.227 Kg, 133.8% theoretical) contaminated with silyl-related by-products. ²MET/PR/0344

### Stage 3: (5-Fluoro-2-methyl-3-quinolin-2-ylmethylindo-1-yl)-acetic acid

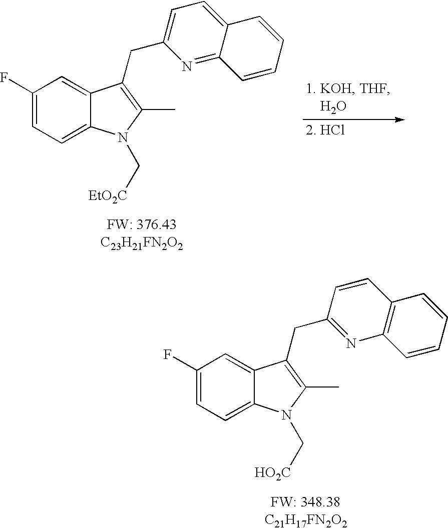

For the purposes of the Stage 3 input calculations, it was assumed that the Stage 2 reaction had progressed in 100% theoretical yield.

Potassium hydroxide (0.486 Kg, 0.53 wt) as a solution in water (5.5 L, 6 vol) was added to a solution of (5-fluoro-2-methyl-3-quinolin-2-ylmethyl-indo-1-y)l-acetic acid ethyl ester (0.916 Kg assumed, 2.44 mol, 1 wt) in tetrahydrofuran (3.66 L, 4 vol) such that the reaction mixture was allowed to exotherm to 30 to 35° C. The reaction was maintained at 30 to 35° C. for 2 h after which time TLC³ analysis (ethyl acetate:toluene 1:1; visualisation: UV) indicated reaction completion by the absence of starting material. tert-Butyl methyl ether (4.6 L, 5 vol) was added and the phases separated such that interfacial material was retained with the aqueous phase. The aqueous layer was washed further with tert-butyl methyl ether (4.6 L, 5 vol), concentrated under vacuum at 35 to 40° C. (water bath) for up to 1 h to remove residual organics and then cooled to 15 to 25° C. The resulting slurry was acidified with aqueous hydrochloric acid (2M, 3.44 L, 3.75 vol) to pH 5.5 such that the temperature was maintained in the range 20 to 25° C. (noted that the solution turned a deep red colour on acidification). The slurry was aged for 1 hour at 15 to 25° C., the pH confirmed as 5.5, the slurry filtered (slow) and the collected solids washed with water (1×1 vol, 1×0.92 L). The wet-cake was azeo-dried with toluene (35 L) until the water content was 0.3% by Karl Fisher analysis affording the crude product as a purple solid (0.767 Kg, 90.5% theoretical corrected for 5.6% w/w toluene). ³Reaction mixture diluted with THF:water prior to analysis

### EXAMPLE 2

### Solubility of Compound 1 Free Acid

In order to provide information on the intrinsic solubility of the unionized form of Compound 1 and the potential increase/decrease in solubility that could be obtained from salt formation a basic solubility screen was carried out. 50 mg of Compound 1 was charged to a vial along with 20 vol of a given solvent. The mixture was stirred at 15 to 25° C. and if a clear solution was obtained then more solid was added until the solution was fully saturated. If a solution was not obtained then the mixture was heated with stirring to reflux and if necessary another 20 vol of solvent was added. DMSO, NMP and DMF mixtures were heated to 100° C. The mixture was then cooled to 15 to 25° C. Table 1 below summarizes the results.

TABLE 1

| | Solution Yes/No | | | Solution on |
|---|---|---|---|---|
| Solvent | 20 vol R.T. | 20 vol reflux | 40 vol reflux | cooling to R.T. |
| Water | No | No | No | No |
| Methanol | No | No | No | No |
| Ethanol | No | No | No | No |
| IPA | No | No | No | No |
| Acetone | No | No | No | No |
| Chloroform | No | No | No | No |
| Acetonitrile | No | No | No | No |
| Ethyl acetate | No | No | No | No |
| Toluene | No | No | No | No |
| Heptanes | No | No | No | No |
| DMSO | No | Yes | n/a | No |
| NMP | No | Yes | n/a | Yes |
| TBME | No | No | No | No |
| DMF | No | No | Yes | No |

R.T. = room temperature

The results showed that Compound 1 is very insoluble (<25 mg/ml) in a variety of solvents. Only NMP retained 50 mg of Compound 1 in 20 vol (i.e. ≧50 mg per ml) at 15 to 25° C. (after obtaining a solution at 100° C.).

### COMPARATIVE EXAMPLE 3

### Attempted Salt Formation Using Conventional Method

The initial screening of bases was done using glass 96 well plates in order to achieve a high throughput so as to allow each combination of base and solvent to be investigated. The technique involves dissolving the sample in a solvent and adding a fixed volume (containing 1 mg) of the resulting solution to each well. Stock solutions of the bases were prepared and a stoichiometric amount was charged to the wells such that each line in the x direction was one particular base except for the 8^{th} line which was left blank. Different potential crystallizing solvents were then added to each row in the y direction (FIG. 1). The plate was then inspected for crystal formation using an inverted microscope.

A wide selection of solvents covering the polarity range from water to heptanes were chosen for the initial screen in order to investigate the solvent effects on crystallisation of the salts. The following solvents were used:
Water, methanol, ethanol, 2-propanol (IPA), acetonitrile (MeCN), tetrahydrofuran (THF), ethyl acetate, (EtOAc) dichloromethane (DCM), toluene, tert-butylmethylether (TBME), acetone, heptanes.

The bases chosen for the screen were selected from the standard list of pharmaceutically accepted salt forming reagents (source: Handbook of Pharmaceutical Salt Properties, Selection and use, edited by P Heinrich Stahl and Camille G Wermuth; Wiley-VCH; ISBN 3-906390-26-8).

The bases were divided into three classes based on the following criteria:

### Class 1 Bases

The class 1 bases are those that are of unrestricted use because they form physiologically ubiquitous ions or because they occur as intermediate metabolites in biochemical pathways. Table 2 shows a list of class 1 bases, their pKₐ values and the composition of the stock solutions used in the experiments described below.

TABLE 2

| | pKₐ Value | | | | |
|---|---|---|---|---|---|
| Class 1 Base | pKₐ 1 | pKₐ 2 | pKₐ 3 | Stock 1 | Stock 2 |
| Ammonium hydroxide | 9.3 | | | 2.15 ml in 100 ml | 0.2 ml in 5 ml |
| 13.36M aq. soln. | | | | H₂O | NMP |
| Choline | >11 | | | n/a | n/a |
| Calcium acetate | 12.6 | | | n/a | 45.4 mg/ml H₂O |
| N-methyl Glucamine, | 8 | | | 56.0 mg/ml H₂O | 56.0 mg/ml NMP |
| Lysine | 10.8 | 9.2 | 2.2 | 42.0 mg/ml H₂O | n/a |
| Magnesium acetate | 11.4 | | | 61.5 mg/ml H₂O | 61.5 mg/ml NMP |
| Potassium hydroxide | 14.0 | | | 18.9 mg/ml H₂O | n/a |
| Sodium hydroxide | 14.0 | | | 11.5 mg/ml H₂O | n/a |

N.B. Potassium hydroxide assumed to be 85% w/w. For Stock 2, 7N Ammonia in MeOH was used.

### Class 2 Bases

The class 2 agents are considered those that are not naturally occurring. However, so far during their profuse application, they have shown low toxicity and good tolerability. Table 3 shows a list of class 2 bases, their pKa values and the composition of the stock solutions used in the experiments described below.

TABLE 3

| Class 2 Base | pKₐ 1 | Stock 1 |
|---|---|---|
| Betaine | 12.2 | 33.6 mg/ml MeOH |
| Deanol | 8.8 | 25.6 mg/ml NMP |
| Diethylamine | 10.9 | 21.0 mg/ml NMP |
| Diethylaminoethanol | 9.6 | 33.6 mg/ml NMP |
| 4-(2-Hydroxyethyl)morpholine | 7.4 | 37.7 mg/ml NMP |
| 1-(2-Hydroxyethyl)pyrrolidine | 9.4 | 33.1 mg/ml NMP |
| Tromethamine (TRIS) | 8 | 34.8 mg/ml NMP |

### Class 3 Bases

Class 3 bases are those that might be interesting under particular circumstances or for solving particular problems. Some are assigned to this class because they have their own pharmacological activity and some have been used much less frequently in the past. Table 4 shows a list of class 3 bases, their pKa values and the composition of the stock solutions used in the experiments described below.

TABLE 4

| | pKₐ Value | | | |
|---|---|---|---|---|
| Class 3 Base | pKₐ 1 | pKₐ 2 | pKₐ 3 | Stock 1 |
| Ethanolamine | 9.5 | | | 17.5 mg/ml THF |
| Ethylenediamine | 10.1 | 7 | | 17.3 mg/ml THF |
| Imidazole | 7 | | | 19.5 mg/ml THF |
| Piperazine | 9.8 | 5.7 | | 24.7 mg/ml THF |
| Triethanolamine | 7.8 | | | 42.8 mg/ml THF |
| Zinc acetate | 14 | | | 52.7 mg/ml NMP |

### General Procedure

In order to charge 1 mg quantities to a 96-well plate it is necessary to make a solution of Compound 1 and then add appropriate portions of this solution to the plate. It would be desirable to use a volatile solvent and subsequently evaporate this to leave the 1 mg portions in the wells. Unfortunately the poor solubility of Compound 1 in volatile solvents did not allow the above method to be followed exactly. The following alternative loading procedure was applied:—

200 mg of free acid were dissolved in 5 ml of NMP to give a 40 mg/ml stock solution. 25 μl of the stock solution was added to each of the 96-wells—which in effect gave 1 mg of Compound 1 per well. 200 μl of solvent was then added to the appropriate wells along with 10 μl of stock base solution (Composition of stock base solutions is shown in Tables 2-4) to give a 1:1 acid:base stoichiometry. The 96-well plates were then shaken at room temperature and visualised after 1 hour and 18 hours using an inverted microscope with crossed polars to assess the degree of crystallinity of any solid present and provide a relative estimate of the quantity of the material present. The individual 96 wells were ranked on a 1 to 5 scale where 1=no crystals/clear solution to 5 being lots of crystals (such that the light from the microscope was almost obscured).

### Class 1 Bases

The screen on the class 1 bases was carried out according to the above procedure. The results appeared flawed as the blank row (no base added) scored highly for crystal growth. In all cases except THF the addition of solvent had caused the precipitation of crystalline Compound 1.

The screen was repeated but this time the bases were added to the NMP solutions of Compound 1 in each well along with 10 μl of water in the blank row and shaken for 30 minutes before adding the solvents. Inspection of the plate prior to solvent addition showed that there were crystals in the blank row. Again the results were unreliable as the crystal formation was just as likely to be precipitation of Compound 1 by water (from the base solutions) as being salts.

The experiment was repeated again but the base solutions were made up in NMP rather than water. Unfortunately it was not possible to prepare solutions of sodium hydroxide, potassium hydroxide or Lysine. Inspection of the plate after 2 hours of shaking the plate with just Compound 1 and base showed no crystals present. The appropriate solvents were then added and the plate inspected after a further 1 hour and 18 hours. Once again the blank row showed the presence of crystals except for the heptanes well (in this case a two phase mixture resulted and subsequently no precipitation occurred).

### Class 2 Bases

The class 2 base counter ion screen was carried out according to the method used in the third run of the class 1 bases (NMP solutions of Compound 1 charged to wells, NMP solutions of bases charged to wells, shaken for 1 hr, solvents charged, inspected after 1 hr and 18 hrs). As in the case of the class 1 bases there were no crystals/salts visible in the wells after shaking the plate for 1 hr with just Compound 1 and the base. One hour after the solvents were added there were crystals in all the betaine, 4-(2-hydroxyethyl)morpholine and the blank wells. The other wells showed little to no crystals.

In order to assess whether salt formation had occurred these reactions were scaled up. For each base/solvent combination 50 mgs of Compound 1 was charged to a vial and dissolved in 25 vols NMP. A solution of the base in 10 vols NMP was charged to the vial such that the stoichiometry of base to Compound 1 was 1:1. The vials were shaken for 1 hour at 15 to 25° C. and then the appropriate solvents (200 vols) charged. After shaking the vials for 18 hours they were examined. Any precipitated solid was collected by filtration and analyzed by 1H NMR. The results showed that no salts were formed for any of the base/solvent combinations—the scaled up samples either precipitated Compound 1 or gave no precipitate.

### Class 3 Bases

The class 3 base screen was carried out as in the class 2 base screen. Once again the results were difficult to interpret. The imidazole and triethanolamine rows showed the presence of crystals as soon as the solvents were added. The ethanolamine and zinc acetate rows gave virtually no crystals in the wells. No conclusions were drawn from this experiment.

### Scale Up

The results from the 96-well plate experiments were inconclusive. The root of the problem stemmed from the insolubility of Compound 1. Also the 96-well plate experiments were carried out at ambient temperature which may have had an impact on any reaction taking place between base and Compound 1.

### EXAMPLE 4

### Formation of Salts of Compound 1

As set out in Example 1, the synthesis of Compound 1 involves an ester hydrolysis at the final stage to give the carboxylic acid. This is carried out using 3 equivalents of potassium hydroxide as base in THF/water. It is evident that a potassium salt must be formed during the hydrolysis. With this in mind 1 g of Compound 1 was charged to a vial along with 3 equivalents of potassium hydroxide. Water (20 vols) was added and the mixture heated to 50° C. to almost give a solution. Upon cooling to 15 to 25° C. a solid precipitated which was collected by filtration. ¹H NMR analysis confirmed that a salt had been formed. This was repeated using 3 equivalents of sodium hydroxide but upon isolation a sticky solid was collected which dissolved when washed with ethanol.

The experiments were repeated using acetonitrile as solvent with a couple of drops of water to help dissolve the base. Two equivalents of base were used this time and both reactions gave their corresponding salts.

Based on the success of this method all the remaining bases were re-screened as follows. 500 mg of Compound 1 was charged to vial along with 2 equivalents of base. 10 volumes of acetonitrile were added (when the base appeared to be insoluble 1 volume of water was also added). The mixtures were heated to 50° C. for 10 minutes and then cooled to 15 to 25° C. Any precipitate was collected by filtration and washed with 5 volumes of acetonitrile before being dried on the filter. The results are presented in Table 5.

TABLE 5

| | pKa of | Solⁿ at | | |
|---|---|---|---|---|
| Base | base | 50° C.? | Yield | Salt? |
| Potassium | 14.0 | Almost | 57% | Yes |
| Sodium | 14.0 | Almost | 69% | Yes |
| Choline | >11 | Yes | — | No precipitate |
| Ammonia | 9.3 | No | 73% | Yes |
| Lysine | 10.8 | No | 131% | Yes |
| N-methyl-D-glucamine | 8 | No | — | Gel resulted, not |
| | | | | isolated |
| Magnesium acetate | 11.4 | No | 137% | Yes |
| Betaine | 12.2 | No | — | No |
| Deanol | 8.8 | No | — | No |
| Diethylamine | 10.9 | No | 83% | Yes |
| Diethylaminoethanol | 9.6 | No | — | No |
| TRIS | 8 | No | 110% | Yes |
| 4-(2- | 7.4 | No | — | No |
| hydroxyethyl)morpholine | | | | |
| 1-(2- | 9.4 | Almost | — | Yes but isolated |
| hydroxyethyl)pyrrolidine | | | | as an oil |
| Piperazine | 9.8 | Almost | 72% | Yes |
| Imidazole | 7 | No | — | No |
| Zinc acetate | 14 | No | 161% | Yes |
| Triethanolamine | 7.8 | No | — | No |
| Ethylenediamine | 10.1 | Yes | 68% | Yes |
| Calcium acetate | 12.6 | No | 153% | Yes |
| Ethanolamine | 9.5 | Yes | 83% | Yes |

Although 13 salts were produced in the screen it was decided only to analyze 9 of them further. The 1-(2-hydroxyethyl)pyrrolidine salt was not picked as it did not form a solid. The magnesium, calcium and zinc salts were also rejected as they formed thick pastes in the reaction vials that were difficult to filter.

The 9 salts chosen for further studies were potassium, sodium, ammonium, lysine, diethylamine, TRIS, piperazine, ethylenediamine and ethanolamine. ¹H NMR showed 1:1 stoichiometry between Compound 1 and the base and the majority had very clean profiles. The Lysine and TRIS salts were not as clean and the spectra suggested that excess base was likely to be present (this was also indicated by >100% yields for these two salts).

### EXAMPLE 5

### Solubility of Salts of Compound 1

Solubility of the salts in water was determined by HPLC. Two standard solutions A and B of Compound 1 were prepared. These two solutions were further diluted twice to give six solutions of decreasing concentration of Compound 1. The six solutions were analyzed by HPLC and a graph of area vs weight was plotted.

Salts were charged to a vial along with HPLC grade water to give a concentration of ˜100 mg/ml. The mixtures were stirred for 18 hours at 15 to 25° C. and then filtered through Whatman™ 1.0 μm PTFE membrane filters. 50 μl of each filtrate was charged to a 10 ml volumetric flask and the volume was made up to 10 ml with the sample diluent. The samples were then analyzed by HPLC.

By using the graph plotted from the standard solutions it was possible to calculate the amount of Compound 1 in the samples and thus the solubility. The results are listed in Table 6 below along with the pHs of the filtered mixtures.

The results show that all the salts are more soluble than Compound 1 in water. The sodium salt is clearly the most soluble but the ethanolamine and piperazine salts also have much improved solubility as well (>50 mg/ml). The pHs of the solutions of the salts were mainly in the range 8 to 9 although the ethylenediamine and potassium salts gave very basic solutions (pH 12).

TABLE 6

| Salt | pH | Solubility mg/ml |
|---|---|---|
| Compound 1 | 7 | 0.05 mg/ml |
| Potassium | 12 | 32.36 mg/ml |
| Sodium | 9 | 84.28 mg/ml |
| Ammonium | 8 | 4.98 mg/ml |
| Lysine | 9 | 9.98 mg/ml |
| Diethylamine | 9 | 6.94 mg/ml |
| Tris | 9 | 3.26 mg/ml |
| Piperazine | 9 | 65.90 mg/ml |
| Ethylenediamine | 12 | 3.44 mg/ml |
| Ethanolamine | 9 | 66.58 mg/ml |

1. A potassium, sodium, ammonium, lysine, diethylamine, tromethamine (TRIS), piperazine, ethylenediamine or ethanolamine salt of a compound of general formula (I): 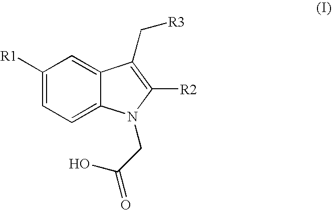
wherein R¹ is halo or cyano;
R² is C₁-C₄ alkyl; and
R³ is quinolyl or phenyl substituted with methane sulfonyl.

2. A salt according to claim 1 which is a potassium salt, a sodium salt, an ethanolamine salt or a piperazine salt.

3. A salt according to claim 1 wherein, in the compound of general formula (I)
R¹ is fluoro.

4. A potassium, sodium, ammonium, lysine, diethylamine, TRIS, piperazine, ethylenediamine or ethanolamine salt according to claim 1, wherein said compound of general formula (I) is
(5-fluoro-2-methyl-3-quinolin-2-ylmethyl-indol-1-yl)acetic acid; or [5-fluoro-3-(4-methanesulfonylbenzyl)-2-methyl-indol-1-yl]acetic acid.

5. A process for the preparation of a salt according to claim 1, comprising:
adding to about 8 to 20 volumes of acetonitrile and about 2 to 3 molar equivalents of a base to a compound of general formula (I) to form a mixture;
optionally adding to the mixture sufficient water to dissolve the compound of general formula (I);
heating the mixture to between about 40 and about 60° C.;
allowing the mixture to cool to about 15 to 25° C.; and
collecting the precipitated salt.

6. A process according to claim 5, wherein the base is ammonium hydroxide, lysine, potassium hydroxide, sodium hydroxide, diethylamine, ethanolamine, ethylenediamine, piperazine or tromethamine (TRIS).

7. A process according to claim 5 wherein, about 10 volumes of acetonitrile are added to the compound of general formula (I).

8. A process according to claim 5 wherein, about 2 molar equivalents of base are used.

9. An aqueous solution comprising at least 3 mg/ml of a potassium, sodium, ammonium, lysine, diethylamine, tromethamine (TRIS), piperazine, ethylenediamine or ethanolamine salt of a compound of general formula (I): 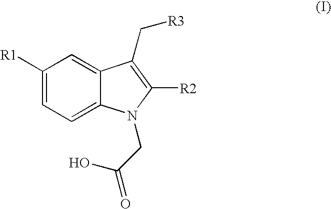
wherein R¹ is halo or cyano;
R² is C₁-C₄ alkyl; and
R³ is quinolyl or phenyl substituted with methane sulfonyl.

10. An aqueous solution according to claim 9, comprising at least 10 mg/ml of a potassium, sodium, piperazine or ethanolamine salt of a compound of general formula (I).

11. An aqueous solution according to claim 10 comprising at least 30 mg/ml of a salt of general formula (I).

12. (canceled)

13. A method of treatment or prevention of a condition or disease selected from the group consisting of allergic asthma, perennial allergic rhinitis, seasonal allergic rhinitis, atopic dermatitis, contact hypersensitivity (including contact dermatitis), conjunctivitis, eosinophilic bronchitis, food allergies, eosinophilic gastroenteritis, inflammatory bowel disease, ulcerative colitis, Crohn's disease, mastocytosis, autoimmune diseases, hyper IgE syndrome, systemic lupus erythematus, psoriasis, acne, multiple sclerosis, allograft rejection, reperfusion injury, chronic obstructive pulmonary disease, rheumatoid arthritis, psoriatic arthritis, osteoarthritis, neurodegenerative diseases, Alzheimer's disease, Parkinson's disease, stroke and amyotrophic lateral sclerosis,
comprising administering to a human an effective amount of a salt according to claim 1.

14. (canceled)

15. A pharmaceutical composition comprising a potassium, sodium, ammonium, lysine, diethylamine, tromethamine (TRIS), piperazine, ethylenediamine or ethanolamine salt of a compound of general formula (I): 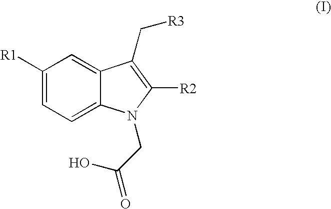
wherein R¹ is halo or cyano;
R² is C₁-C₄ alkyl; and
R³ is quinolyl or phenyl substituted with methane sulfonyl
together with a pharmaceutically acceptable excipient or carrier.

16. A pharmaceutical composition according to claim 15 formulated for oral, nasal, bronchial or topical administration.

17. A composition according to claim 15, further including one or more additional active agent useful in the treatment of diseases mediated by PGD₂ at the CRTH2 receptor.

18. A composition according to claim 17, wherein the one or more additional active agent is selected from a group consisting of:
β2 agonists;
corticosteroids;
antihistamines;
leukotriene antagonists;
anti-IgE antibody therapies;
anti-infectives;
anti-fungals; immunosuppressants;
other antagonists of PGD₂ acting at receptors other than CRTH2; inhibitors of phosphodiesterase type 4 ;
drugs that modulate cytokine production;
drugs that modulate the activity of Th2 cytokines IL-4 and IL-5;
PPAR-γ agonists; and
5-lipoxygenase inhibitors.

19. A process for the preparation of a pharmaceutical composition according to claim 15, comprising bringing a potassium, sodium, ammonium, lysine, diethylamine, tromethamine (TRIS), piperazine, ethylenediamine or ethanolamine salt of a compound of general formula (I) in into conjunction or association with a pharmaceutically or veterinarily acceptable vehicle.

20. A method of treatment or prevention of a condition or disease selected from the group consisting of allergic asthma, perennial allergic rhinitis, seasonal allergic rhinitis, atopic dermatitis, contact hypersensitivity (including contact dermatitis), conjunctivitis, eosinophilic bronchitis, food allergies, eosinophilic gastroenteritis, inflammatory bowel disease, ulcerative colitis, Crohn's disease, mastocytosis, autoimmune diseases, hyper IgE syndrome, systemic lupus erythematus, psoriasis, acne, multiple sclerosis, allograft rejection, reperfusion injury, chronic obstructive pulmonary disease, rheumatoid arthritis, psoriatic arthritis, osteoarthritis, neurodegenerative diseases, Alzheimer's disease, Parkinson's disease, stroke and amyotrophic lateral sclerosis, comprising the simultaneous, separate or sequential administration of a salt of a compound of general formula (I) and one or more of the additional active agents according to claim 18.

21. A method according to claim 13 comprising administering to a human or an animal, a composition containing a salt of general formula (I), wherein said composition comprises an additional active agent useful for the treatment of diseases and conditions mediated by the action of PGD₂ at the CRTH2 receptor.

22. A method according to claim 21, wherein the additional active agent is selected from a group consisting of:
β2 agonists;
corticosteroids;
antihistamines;
leukotriene antagonists;
anti-IgE antibody therapies;
anti-infectives;
anti-fungals;
immunosuppressants;
other antagonists of PGD₂ acting at other receptors;
drugs that modulate cytokine production;
drugs that modulate the activity of Th2 cytokines IL-4 and IL-5;
PPAR-γ agonists; and
5-lipoxygenase inhibitors.

23. A salt according to claim 1, wherein in the compound of formula (I) R² is methyl.

24. A salt according to claim 1, wherein in the compound of formula (I) R³ is 2-quinolyl or 4-methanesulfonylphenyl.
